(19) United States
(12) Patent Application Publication
(10) Pub. No.: US 2009/0035745 A1
(43) Pub. Date: Feb. 5, 2009

(54) Site-Specific Dosing of Cellular Cultures

(76) Inventors: Jason B. Shear, Austin, TX (US); Rex Nielson, Austin, TX (US)

Correspondence Address: BAKER BOTTS, LLP, 910 LOUISIANA, HOUSTON, TX, 77002-4995, US

(21) Appl. No.: 12/185,184

(22) Filed: Aug. 4, 2008

Related U.S. Application Data
(60) Provisional application No. 60/953,759, filed on Aug. 3, 2007.

(51) Int. Cl. C12Q 3/00; C12M 3/00

(52) U.S. Cl. 435/3; 435/286.5

(57) ABSTRACT

Systems and methods for site-specific dosing of cellular cultures are provided. Some of the systems provided comprise at least two laminar flow channels; and an ablatable membrane that is disposed between the at least two laminar flow channels. Other systems provided comprise at least two laminar flow channels; and an ablated membrane that is disposed between the at least two laminar flow channels. Some of the methods provided comprise providing at least two laminar flow channels; providing a membrane disposed between the at least two laminar flow channels, wherein the membrane is selected from the group consisting of an ablatable membrane and an ablated membrane; placing cells into at least one of the at least two laminar flow channels; providing a reagent medium; and flowing the reagent medium through at least one other of the at least two laminar flow channels.

### CROSS-REFERENCE TO RELATED APPLICATIONS

This application claims the benefit of U.S. Provisional Patent Application Ser. No. 60/953,759, filed Aug. 3, 2007, the entire disclosure of which is hereby

### STATEMENT OF GOVERNMENT INTEREST

This invention was developed with support under Grant Number 0317032 awarded by the National Institutes of Health. The U.S. Government has certain rights in the invention.

### BACKGROUND

Controlled chemical dosing of cells in culture is an indispensable tool in the study of cell differentiation, growth, activity, and death. Agents for selectively perturbing second-messenger signaling, gene-transcription rates, and metabolic processes are used to probe cellular function in a defined fashion (1, 2). Although entire culture dishes commonly are dosed with a reagent via perfusion or pipet, there is growing understanding for a need to exert greater spatio-temporal control over interactions between chemicals and cells. In particular, studies focused on signaling within and between individual cells, chemotaxis, and neuritic pathfinding have benefited from a battery of methods for creating localized chemical signals. In addition, the ability to deliver labeling reagents to subcellular domains has the potential to clarify mechanisms involved in processing and transport of macromolecules and organelles (3, 4).

Various strategies have been used to locally target chemical agents within cell-culture environments. Of these, puffer-pipet expulsion and photolytic cleavage of caged cellular effectors both have been adopted as relatively routine tools for dosing with subcellular resolution. While both methods present important capabilities, each suffers from serious limitations. Micropositioned puffer pipets can be used to accurately target nanoliter volumes to subcellular coordinates and, consequently, have been used extensively in studies of polarized cellular responses, including growth-cone turning and localized stimulation of neurons (5, 6). It generally is not feasible, however, to independently position more than several pipets in tandem or to rapidly reposition fragile tips to new sites of interest. For chemical uncaging, dosing volumes can be as small as ˜1 fL and the site at which effectors are photolytically created can be rapidly selected and changed in a manner unattainable with puffer pipet (7, 8). Nevertheless, the applicability of uncaging is severely limited by the need to synthesize a new caged precursor for every effector of interest, many of which cannot be caged using current strategies. For dosing using either micropipets or uncaging, it typically is not feasible to establish steep concentration gradients that are sustainable for extended periods.

Microfluidic technologies have been used control chemical gradients in microfabricated chambers amenable to cell culture (9-12). Whitesides and coworkers developed a method in which parallel laminar-flow streams formed within a microfluidic environment could be used to dose subcellular regions of cells with reagent (13, 14) In this approach, the interfacial region between streams remains sharp, with mixing across stream boundaries limited to the small amount of diffusional transport that occurs as streams rapidly traverse a confluence channel. Unlike dosing using micropipets or uncaging, steep gradients can be easily established and sustained indefinitely.

Despite its advantages, the microfluidic approach has important limitations. Most significantly, the complexity of the chemical environment is constrained by the initial design of the microfluidic device, and, as with the use of puffer pipets, practical considerations probably limit simultaneous dosing to a few independent subcellular sites. Moreover, although stream-interface positions can be manipulated laterally within a confluence channel, stream directionality must be oriented along the channel's longitudinal axis. As a consequence, a structure such as a neurite growing longitudinally in a channel would either have to be dosed along its entire extent or not at all.

To characterize the role of spatially heterogeneous signaling in cellular function, methods are required for differentially exposing distinct regions of individual cells to externally applied reagents. Although a range of standard approaches exist for generating localized chemical gradients in culture, including puffer-pipet spritzing and photolytic release of caged effectors, each are limited in key respects.

### SUMMARY

The present disclosure, according to certain embodiments, relates to a cell-dosing strategy that addresses these limitations, providing the means to create steep gradients of any aqueous-miscible compound at essentially unlimited numbers of sites in parallel.

The present disclosure provides, in certain embodiments, a system for site-specific dosing of cellular cultures, the system comprising: at least two laminar flow channels; and an ablatable membrane that is disposed between the at least two laminar flow channels.

The present disclosure provides, in certain embodiments, a system for site-specific dosing of cellular cultures, the system comprising: at least two laminar flow channels; and an ablated membrane that is disposed between the at least two laminar flow channels.

The present disclosure provides, in certain embodiments, a method for site-specific dosing of cellular cultures comprising the steps of: providing at least two laminar flow channels; providing a membrane disposed between the at least two laminar flow channels, wherein the membrane is selected from the group consisting of an ablatable membrane and an ablated membrane; placing cells into at least one of the at least two laminar flow channels; providing a reagent medium; and flowing the reagent medium through at least one other of the at least two laminar flow channels.

In one approach, cells are cultured on a micron-thick polymer membrane that serves as a barrier between two stacked laminar-flow channels: one containing the cell culture and the other serving as a reagent flow cell. By focusing a pulsed laser beam onto one or more selected membrane positions, micron-diameter pores can be ablated upstream of desired cellular targets. Nascent pores thus serve as ports-of-entry into the culture environment for reagent streams capable of modifying subcellular features at positions potentially hundreds of microns from ablation sites. Importantly, individual reagent streams also can be rapidly eliminated by photocrosslinking a protein plug over a selected pore. This versatile strategy for dynamically reshaping the chemical microenvironments in which cells reside should be useful in a broad range of cell-biology applications, ranging from neurotrophic modulation of neurite pathfinding to stimulation of cellular networks.

The features and advantages of the present invention will be readily apparent to those skilled in the art upon a reading of the description of the embodiments that follows.

### DRAWINGS

Some specific example embodiments of the disclosure may be understood by referring, in part, to the following description and the accompanying drawings.

FIG. 1 shows a strategy for producing laminar-flow dosing streams via ablation of polymer membranes. (a) PDMS-device design. Cells are cultured on a several-micron-thick membrane, which is inserted into the device with the cells on the underside of the membrane facing a microscope objective. A femtosecond pulsed laser beam (red cone) is focused by the objective onto the membrane to create an ablation pore at a desired position. Effector solution from the higher pressure top flow channel is forced through the pore, producing a tightly defined reagent stream that propagates across the membrane surface within the cell-culture environment. (b) Scanning electron micrographs (SEMs) of ablation pores created in 6-μm-thick polycarbonate membranes showing oblong damage regions surrounding central pores. The scale bar for the left image is 4 μm. An array of pores is shown to the right. (c) A lower-magnification SEM showing the more symmetric and smaller pores created in 2.5-μm-thick Mylar. Scale bar, 2 μtm.

FIG. 2 shows the formation of laminar-flow streams through photoablated pores. (a) Confocal fluorescence microscopy of 1-μM fluorescein streams emerging through pores created in a 6-μm-thick polycarbonate membrane. The image in the right panel was created by summing a stack of confocal images ranging from the membrane to tens of microns into solution. Measurements were acquired using a 20×, 0.7 NA air objective with 0.34 μm steps between stack images. The panel at the upper left plots intensity along a line running from the top of the image downward through the center of the middle plume. Fluorescein intensity decreases by ˜20% over the 350-μm length of stream shown. The plot on the lower left shows intensity along transverse lines running left-to-right across the image at two positions downstream from the pores (87 and 347 μm). Note the relatively minor increase in stream widths at the more distal position. (b) Differential-interference-contrast image (inset) and fluorescence intensity surface plot demonstrating the use of a series of pores to dose an extended region of a cultured cell. A neurite and varicosity region (asterisk, both images) of an NG108-15 cell cultured on a polycarbonate membrane is labeled with CFDA (50 μM) over ˜75 μm using a set of six pores. Flow direction is represented by the downward arrow (inset). The varicosity region is labeled most intensely, as it lies both in the direct path of the dye stream and is significantly larger in volume than the neurite. HBS buffer was flowed through the cell chamber at a volume flow rate of 0.15 mL min⁻¹. Scale bar (inset), 30 μm.

FIG. 3 shows the closure of pores using protein photocrosslinking. Left panel: An SEM of a bovine serum albumin (BSA) plug fabricated to obstruct a pore formed in Mylar. Scale bar, 5 μm. Right five panels: Sequential plugging of laminar-flow BSA streams (1-4) in a culture of NG108-15 cells using multiphoton-excited protein photocrosslinking. FAD was used as a non-toxic photosensitizer to promote BSA crosslinking at the entrances to pores ablated in a polycarbonate membrane. From left, sequential images were acquired at t=0 s, t=132 s, t=185 s, t=190 s, and t=206 s. HBS buffer was flowed through the cell chamber at a volume flow rate of 0.15 mL min⁻¹. Scale bar, 30 μm.

FIG. 4 shows subcellular labeling of neuronally differentiated cells using multiple laminar-flow streams. (a) NG108-15 dosing using the plasma-membrane-permeant fluorescein dye, CFDA. Four sets of pores are visible in the transmission image (top), with clusters numbered according to the sequence in which they were created. After formation of pore clusters 1-3 in the Mylar substrate, CFDA laminar-flow streams were established to selectively dose subcellular regions; after 1 min. exposures to 50 μM CFDA and dye wash-out, fluorescence data were acquired using wide-field imaging. Fluorescence intensities are displayed as surface plots corresponding to t=0 (top plot, pore cluster 1), after 14.5 min. (middle, pore cluster 2), and after 27 min. (bottom, pore clusters 3). Scale bar, 50 μm. (b) Point labeling of opposing neurites on an NG108-15 cell using Mitotracker Green FM. Two pores (1 and 2) were created in the Mylar substrate, seen as concentric circles (top, transmission image) and fluorescence peaks (bottom, fluorescence intensity surface plot). Subcellular sites labeled by pores 1 and 2 are designated in the fluorescence image as 1′ and 2′, respectively. Fluorescence data were acquired after a 1 min. exposure to 2 μM Mitotracker followed by ˜1 min. of dye wash-out. Scale bar, 10 μm. For the studies shown in parts a and b, HBS buffer was flowed through the cell chamber during the dosing period at rates of 0.15 and 0.45 mL min⁻¹, respectively.

FIG. 5 shows a sequence of selective dehydration of an NG108-15 cell. A laminar-flow ethanol stream was introduced through a pore (asterisk, middle image) formed in a polycarbonate substrate. Based on the direction of flow (arrows, left panel), ethanol was delivered to the cell identified in the final panel (acquired 25 s after pore formation). Throughout dosing, HBS buffer was flowed through the cell chamber at a rate of 0.15 mL min⁻¹. Scale bar, 30 μm.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure provides, in certain embodiments, a system for site-specific dosing of cellular cultures, the system comprising: at least two laminar flow channels; and an ablatable membrane that is disposed between the at least two laminar flow channels.

The present disclosure provides, in certain embodiments, a system for site-specific dosing of cellular cultures, the system comprising: at least two laminar flow channels; and an ablated membrane that is disposed between the at least two laminar flow channels.

The present disclosure provides, in certain embodiments, a method for site-specific dosing of cellular cultures comprising the steps of: providing at least two laminar flow channels; providing a membrane disposed between the at least two laminar flow channels, wherein the membrane is selected from the group consisting of an ablatable membrane and an ablated membrane; placing cells into at least one of the at least two laminar flow channels; providing a reagent medium; and flowing the reagent medium through at least one other of the at least two laminar flow channels.

The ablatable membrane or ablated membrane may be ablated by any directed energy source capable of creating an opening in the membrane at a position such that fluid flow or electrical conductivity through the membrane, or both, is established at the opening. The directed energy source may be a light source, for example, but not limited to, a laser light source. Such lasers may include, but are not limited to, a femtosecond titanium/sapphire or frequency-doubled Q-switched Nd:YAG laser.

The ablatable membrane may comprise any material capable of being ablated using a directed energy source such that an opening can be created that allows fluid flow or electrical conductivity through the membrane, or both, is established at the opening. For example, the ablatable membrane may comprise, but is not limited to, polyethylene terephthalate, polycarbonate, or any derivative thereof. The term “derivative” includes any compound that is made from one of the listed compounds, for example, by replacing one atom in the listed compound with another atom or group of atoms, rearranging two or more atoms in the listed compound, ionizing one of the listed compounds, or creating a salt of one of the listed compounds. The term “derivative” also includes copolymers, terpolymers, and oligomers of the listed compound.

The ablated membrane may comprise any material having a opening that allows fluid flow or electrical conductivity or both at the opening. For example, the ablated membrane may comprise polyethylene terephthalate, polycarbonate, or any derivative thereof.

In some embodiments of the present invention, the systems of the present invention may further comprise cells cultured within at least one of the at least two laminar flow channels. Accordingly, such systems may be used for site-specific dosing of cells. In other embodiments of the system, at least one of the at least two flow channels may not be fully enclosed. In still other embodiments of the system, a port selection valve may be connected to at least one of the at least two laminar flow channels.

The present disclosure also provides, in certain embodiments, an approach for cellular dosing. For example, as shown in Figure la, cells are cultured on an ablatable membrane that separates two stacked laminar-flow chambers. Brief application of a train of focused energy pulses introduces at least one pore in the membrane, producing at least one laminar-flow effector stream that enters the lower-pressure cell-culture chamber. Because membrane ablation may be performed in the presence of one or more cultured cells, features of interest (e.g., cell bodies, synaptic terminals, growth cones) can be specifically targeted for chemical dosing. In contrast to other chemical dosing approaches, this strategy is amenable to creation of essentially unlimited numbers of laminar-flow streams at arbitrarily selected positions. Importantly, the design of this system may be readily modified to provide on-the-fly control of flow directionality and access for patch pipets.

Thus, certain embodiments the present disclosure also relate to a method for site-specific dosing of cellular cultures comprising: providing at least two laminar flow channels; providing an ablatable membrane or ablated membrane disposed between the at least two laminar flow channels; placing cells into at least one of the at least two laminar flow channels; providing a reagent medium; and flowing the reagent medium through at least one other of the at least two laminar flow channels. The ablatable membrane may be ablated by any directed energy source capable of creating an opening in the membrane at a position such that fluid flow or electrical conductivity or both is established at that position. The directed energy source may be a light source, for example, a laser light source. Such lasers may be a femtosecond titanium/sapphire or frequency-doubled Q-switched Nd:YAG lasers. The ablatable membrane may comprise any material capable of being ablated using a directed energy source such that an opening can be created that allows fluid flow or electrical conductivity through the membrane, or both, is established at the opening. For example, the ablatable membrane may comprise polyethylene terephthalate, polycarbonate, or any derivative thereof. The ablated membrane may comprise any material having a opening that allows fluid flow or electrical conductivity or both at the opening. For example, the ablated membrane may comprise polyethylene terephthalate, polycarbonate, or any derivative thereof.

In certain embodiments, the methods of the present invention may additionally comprise the step of at least substantially reducing the transport of material, the conduction of electricity, or both, across the ablatable membrane or ablated membrane. The step of at least substantially reducing the transport of material, the conduction of electricity, or both, across the ablated polymer membrane may be performed by any directed energy source capable of sealing an opening in the membrane at a position such that fluid flow or electrical conductivity or both is restricted at that position. In certain embodiments, the step of at least substantially reducing the transport of material across the ablatable or ablated membrane with a directed energy source may comprise the crosslinking of proteins by the directed energy source.

In certain embodiments of the methods of the present invention, at least one of the at least two flow channels may not be fully enclosed. Further, the methods of the present invention may additionally comprise providing a port selection valve; connecting the port selection valve to at least one of the at least two laminar flow channels; and allowing the port selection valve to control the flow of fluid through at least one of the at least two laminar flow channels.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### EXAMPLES

Device configuration

In the design, a 2-mm by 8-mm field of cells (see below) was sandwiched between two parallel polydimethylsiloxane (PDMS) flow channels. The membrane acted both as a substrate for support of adherent cells and as a barrier between the two flow channels. A standard microscope coverglass served as the bottom of the lower channel. Because the membrane was situated within the working distance of the microscope objective (˜100 μm above the coverslip), it was possible to both image cells and to focus a laser beam on the membrane for ablation. The low profile of the channel also resulted in low Reynolds number (laminar) flow even at the highest flow rates examined, a condition important both for viability of cultured cells and for creating well-defined reagent streams as solution emerges through pores from the upper chamber. The comparatively large width of the channel (2.5 mm) readily accommodated an extended field of cultured cells, providing many potential cellular targets for dosing and the possibility for creating extensively networked neuronal cultures. To enable rapid exchange of solutions for cell dosing, a similar flow-channel design was adopted for the upper chamber. A positive pressure gradient was created between the upper and lower chambers by constricting the drain tube of the upper flow cell, thus ensuring that solution flowed downward through a pore into the cell culture region. Viton tubing (1.6-mm inner diameter; Cole-Parmer, Vernon Hills, Ill.) provided a fluidic connection between the chambers and a syringe pump (BS-9000, Braintree Scientific, Braintree, Mass.) capable of delivering volumes of 6 μL-1300 mL h⁻¹.

Device Fabrication

Flow channels were fabricated using modifications to standard PDMS molding processes (15). To provide a positive relief for the upper channel a strip of transparency film 2.5-mm wide, 22-mm long and 0.1-mm thick was fastened to the bottom of a 5.5 cm diameter polystyrene Petri dish using a thin application of silicone rubber adhesive (RTV 108, GE silicones, Niskayuna, N.Y.). Masters for the drain and feed channels were created with pieces of polyurethane coated wire (0.9-mm diameter) glued next to the ends of the transparency strip in an orientation initially normal to the surface. At a height of ˜3 mm the wires were bent parallel to the surface and extended away from the channel master. PDMS (RTV 615 parts A and B, GE Silicones) was mixed at a 10:1 ratio of monomer to curing agent then degassed by centrifuging at 2000 rpm for 10 min. The mixture then was poured into the Petri dish mold to a height of ˜6 mm and further degassed in a vacuum chamber. PDMS was cured at 70° C. for 1 h.; hardened polymer was separated from its masters/mold and was cut to its final dimensions using a razor. The resulting form was a PDMS block 40-mm long, 14-mm wide and 6-mm high which encased a rectangular channel accessed by additional channels to feed and drain solution.

The lower channel was fabricated in a similar manner except that after the addition of a thin layer of PDMS, a 22-mm by 22-mm No. 1½ coverglass (Erie Scientific Company, Portsmouth, N.H.) was compressed onto the transparency film that formed the master for the lower channel. Additional PDMS mixture was added, burying the coverglass and filling the Petri dish mold to the height of ˜2.5 mm. After curing, PDMS was separated from the Petri dish and the masters/mold. A 15-mm by 15-mm wide window for the microscope objective was made by cutting through and removing the PDMS on the side opposite the channel, thereby exposing part of the coverglass that was embedded in the elastomer. The finished bottom flow channel was 0.1-mm high, 2.5-mm wide and 22-mm long, with the optically transparent coverglass serving as the floor of the channel.

Chemicals

Except where noted, all chemicals were purchased from Sigma-Aldrich (St. Louis, Mo.). Bovine serum albumin (BSA) was obtained from Equitech-Bio (Kerrville, Tex.). Mitotracker Green FM (M7514) was purchased from Molecular Probes, Inc. (Eugene, Oreg.).

Cell Culture

Substrates for cell culture consisted of 2.5-μm thick Mylar polyester membranes (SPI Supplies, West Chester, Pa.) or 6-μm thick polycarbonate membranes (Goodfellow, Devon, Pa.). Pieces of polymer membrane were held taut by taping them to plastic frames having 18-mm wide by 50-mm long rectangular openings. (PDMS) wells were placed in the center of the pieces of membrane, forming a stable seal by means of surface adhesion. The wells created ˜1-mL containers for growth medium. The bottom of the wells had 2-mm by 8-mm rectangular apertures which provided a means to pattern adherent cells onto the polymer membrane. The PDMS well/polymer membrane adhesion structures were washed multiple times with ethanol and de-ionized water. Wells were filled for 20 min. with 33 μg/mL collagen from rat tail (C7661, Sigma) in HEPES-buffered saline (HBS) (10-mM HEPES, 135-mM NaCl, 5-mM KCl, 2-mM MgCl₂, 2-mM CaCl₂, 10-mM D-glucose, pH 7.4) then rinsed several times with plain HBS before the addition of the cell suspension.

Neuroblastoma-glioma (NG108-15) cells were purchased from the American Type Culture Collection (Manassas, Va.) and cultured in DMEM (Mediatech, Inc., Herndon, Va.) supplemented with 10% fetal-bovine serum (FBS), penicillin (100 mg/L), and streptomycin (100 kUnits/L) purchased from Invitrogen (Carlsbad, Calif.). Flasks were maintained at 37° C. in a 10% CO₂ atmosphere with saturated H₂O. Cells were suspended in a low-serum (1% FBS) growth medium at a concentration of ˜30,000 cells/mL, placed into the PDMS wells on the polymer membrane and incubated for 1-3 days in the low-serum medium to induce neuronal differentiation.

Cell Loading

After incubation, cells were loaded into the flow channels by inverting the membrane on which the cells had been plated; the PDMS well then was removed, leaving a hanging drop that bathed the plated cells. The two flow channels were aligned on either side of the cell field and compressed to sandwich the membrane. A block with two screws was used to hold and compress the mated channels onto a heated microscope stage, reinforcing a seal between the channels and the membrane. This process yielded two channels ˜100-μm high and 2.5-mm wide that were separated by a polymer membrane, with a field of cells adhered to the underside of the membrane. The lower channel feed tube was secured to the heated stage and the temperature was adjusted so that solution exiting the lower channel was 37° C.

Membrane Ablation

Pores in membranes were produced by ablation using the output from a femtosecond titanium/sapphire (Ti:S) laser (Coherent Mira, Santa Clara, Calif.) tuned to 750 nm. The laser repetition rate was 76 MHz and the pulse duration was ˜150 fs. The Ti:S beam was aligned into a Zeiss Axiovert (inverted) microscope. The beam was expanded and collimated so that it approximately filled the back aperture of a 40× objective (Olympus UPlanFl, 0.75 NA). The average power of the laser ranged from 200 to 350 mW (measured before the objective), yielding an approximate fluence per laser pulse at the focal spot of ˜3-6 J cm⁻². Laser exposure periods were controlled using a Uniblitz UHS1 shutter (Vincent Associates, Rochester, N.Y.).

Microscopy

Light transmission photomicrographs were obtained on the Axiovert using a cooled CCD camera (C4742-98; Hamamatsu, Bridgewater, N.J.). Fluorescence images were acquired using a Zeiss HBO 100 mercury lamp and a FITC filter set. Confocal images of plumes were obtained using a Leica SP2 AOBS confocal microscope (Wetzlar, Germany). Image analysis was performed using ImageJ (16).

To obtain scanning electron micrographs of ablated pores, polymer membranes were washed with ethanol and air dried. Afterward, membranes were sputter coated with gold to a nominal thickness of 9 nm and imaged using an LEO 1530 SEM (Leo Electron Microscopy Ltd., Zeiss, Oberkochen, Germany).

Membrane Ablation

Various investigators have examined ablation of materials using focused, sub-picosecond laser pulses that promote dielectric breakdown above critical threshold intensities. Although some controversy exists regarding ablation mechanisms, optical breakdown of dielectric materials generally is modeled as proceeding through a self-terminating electron-impact ionization process (17). In a number of instances, laser drilling in polymer membranes has been performed using amplified femtosecond sources, lasers that provide extremely high-energy pulses at much lower repetition rates than conventional femtosecond oscillators. Unlike fluorescence applications, in which it generally is desirable from a signal-to-noise standpoint to excite a fluorophore at rates that approach the inverse of the emitter's excited-state lifetime, for micromachining it often is desirable to use a laser repetition rate low enough for focal heating to dissipate between pulses. When this condition is not met, over many laser pulses an area significantly larger than the laser focal spot can become heated above the material's melting temperature, leading to larger and less uniform features than would be obtained from dielectric breakdown alone. Nevertheless, relatively high-resolution micromachining has been accomplished using high-repetition-rate femtosecond lasers, particularly in cases where the total number of interactive pulses remain small (18).

In the current studies, a 40× air objective was used to create pores in Mylar and polycarbonate membranes that separated adjoining flow cells. Ablation of polymer membranes was accomplished by focusing a high repetition rate (76 MHz) Ti:S beam approximately midway into a membrane. In studies using 6-μm-thick polycarbonate membranes, exposure was limited to a period of ˜150 ms using a laser power of ˜200 mW (measured before the objective). For ablation of 2.5-μm-thick Mylar, various exposure conditions were evaluated to achieve minimum pore sizes and to optimize the regularity and reproducibility of pore shapes. Ultimately, a procedure was adopted in which the membrane was irradiated by a train of ten 1-ms exposures at 350-mW average power, spaced by 50-ms intervals. Insertion of these extended “dark” periods provided an opportunity for the ablation site to dissipate heat between periods of brief and intense irradiation, resulting in more regularly sized pores than were attained using continuous exposure. Pore size does not appear to depend strongly on laser power, so long as laser power is set to a level significantly above a threshold for ablation. Positioning of the laser focus within the membrane likely plays a more significant role in determining pore size, although detailed studies on this effect were not conducted in this initial study.

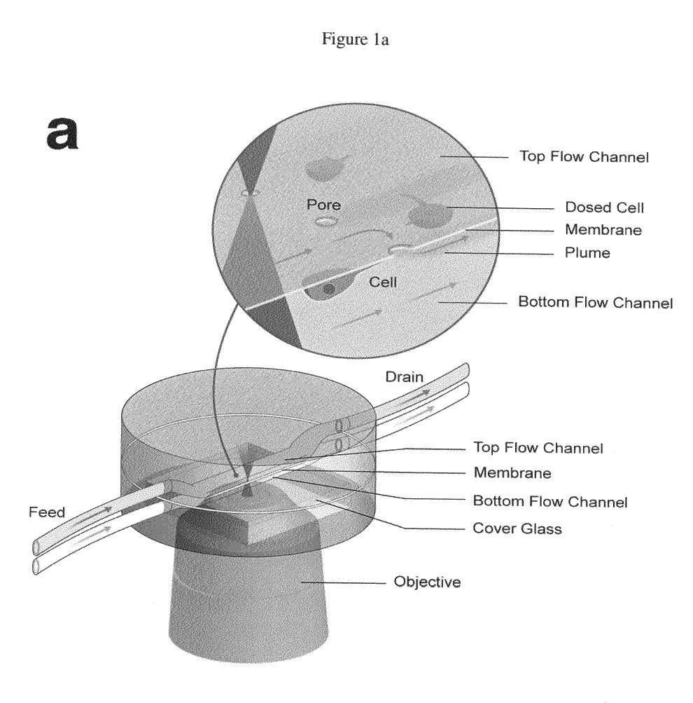

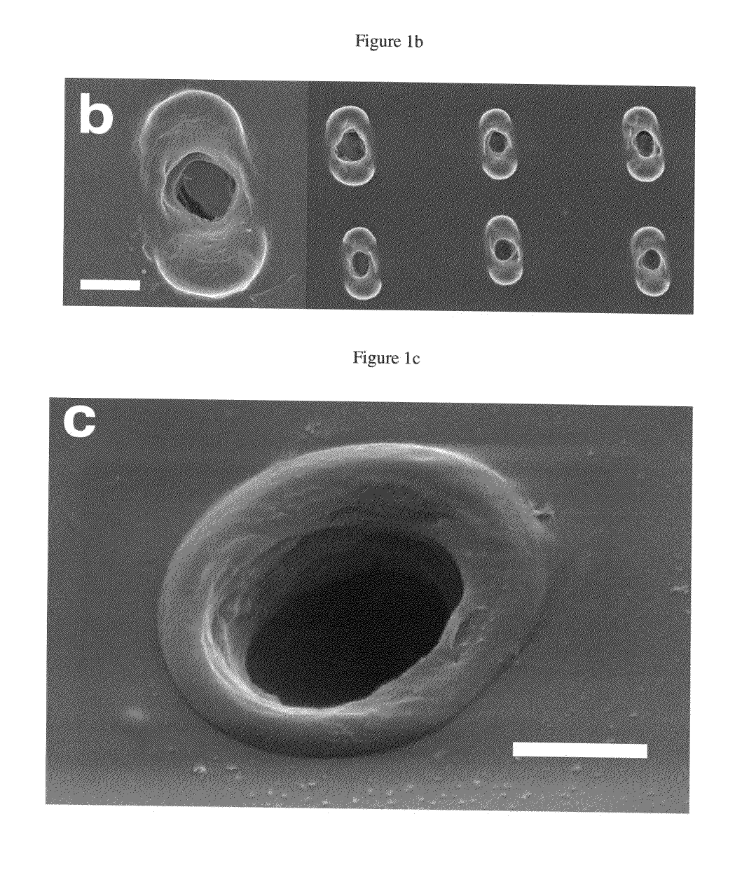

High-resolution analysis of pores formed in polycarbonate and Mylar using these procedures was performed using scanning electron microscopy (SEM). As shown in FIG. 1b, in polycarbonate a somewhat elongated central pore (pore area, 12±4 μm²) is created within an asymmetric lip. The appearance and relatively large diameter of the pores (˜4 μm diameter; more than 10-fold larger than the focal spot diameter) indicate that the ablation process likely involves both dielectric breakdown and an accompanying thermal process (18). The apertures in 2.5-μm thick Mylar (FIG. 1c) were both smaller (pore area, 3.2±0.7 μm²) and less oblong. Standard deviations in membrane pore areas were determined through SEM measurements on series of pores fabricated within single polycarbonate and Mylar membranes. Provided that the laser focal point remained stationary with respect to the membrane during pore ablation, this level of variability appears similar to that fabricated in other membranes (on different days) as observed using optical microscopy.

When ablation is performed with aqueous solutions in both the upper and lower chambers, gas bubbles created by the ablation process often adhere to the lip surrounding the pore, obstructing flow through the aperture. Such blockage can be avoided by filling the upper chamber with air during laser exposure, an approach that requires the chamber to be re-filled with solution after formation of the pore.

Laminar Stream Characteristics

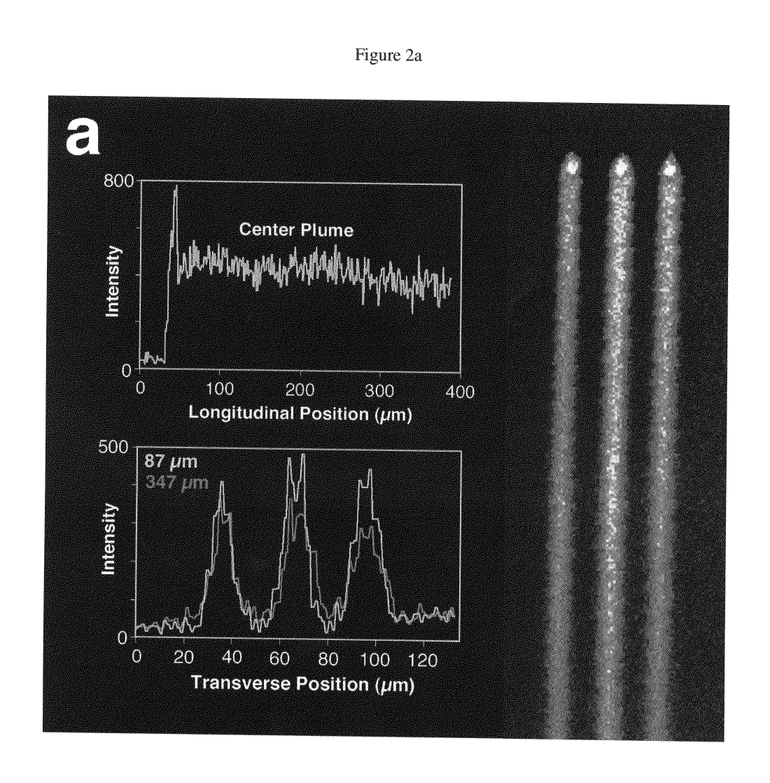

Confocal fluorescence microscopy was used to characterize the three-dimensional properties of laminar-flow streams created by flow of 1-μM fluorescein through representative pores in 6-μm thick polycarbonate membranes. FIG. 2a shows streams emerging from three pores laterally spaced by ˜30 μm. Using a lower-chamber flow rate of 1.0 mL min⁻¹ (15 μm ms⁻¹ at a distance of 4 μm from the membrane), well-defined streams are created that extend from pores for hundreds of microns with minor broadening. The mean 1/e stream radius 10 μm downstream from the pore edge at a nominal distance of 4 μm from the membrane was measured to be ˜6 μm. Laminar-flow streams run parallel to the membrane, with dye detectable at a maximum distance of ˜8 μm from the membrane surface. Within experimental error, the concentration of the dye at the center of the laminar-flow stream 10 μm downstream from the pore was equivalent to the concentration of dye supplied to the upper chamber. As expected, at a lower flow rate (0.15 mL min⁻¹; 2.3 μm ms⁻¹ 4 μm from the membrane), plumes were more diffuse, with fluorescein fanning out significantly tens of microns downstream from the pore. However, within 10 μm of the pore the dye stream remained well defined, with a measured lateral radius of ˜7 μm. As a consequence, specific targets can be dosed with reasonably high resolution even at lower flow rates.

Initial characterization of laminar streams emerging through pores formed in Mylar indicates that stream radii decrease as a consequence of the smaller and more symmetric ablation pores. Using widefield fluorescence to estimate stream dimensions via de-quenching of fluorescein emission (i.e., as a consequence of a pH differential between the two flow channels), 1/e radii of flow streams ˜10 μm downstream from the pore edge are estimated to be smaller than 5 μm.

Notably, the dimensions of laminar-flow streams are consistently larger than their corresponding aperture diameters, even at positions immediately adjacent to pores. Various factors in addition to pore diameter likely influence the concentration distribution and morphology of laminar-flow streams, including the shape of the pore and its surrounding burr, the cross-sectional area of the pore and the pressure drop between the chambers.

In these initial studies, it was not feasible to dictate the diameter of pores with a high degree of control. In both Mylar and polycarbonate, pores having the minimum achievable diameters (FIG. 1b-c) can be created with good reproducibility and over a limited range, pore size can be increased by using larger average laser powers or longer exposure times. Ultimately, however, threshold values are reached at which violent ablation results in membrane cratering, a process that yields apertures with diameters several-fold larger than pores fabricated below threshold. From a utilitarian standpoint, however, we found it feasible to create wider plumes by ablating arrays of densely packed small pores (FIG. 2b).

Changing Flow Directionality

Studies have been performed to develop a flow cell capable of dynamically changing the flow directionality, allowing the orientation of a reagent plume to be targeted to otherwise inaccessible regions of the culture environment. In our initial design, a flow cell was fabricated that consisted of four paired inlet/outlet lines (with dimensions similar to those in the single inlet configuration) arranged in an asterisk-like fashion, allowing inlet and outlet ports to be changed on-the-fly. The master for this flow cell was created by conventional photolithographic methods in which an SU-8 precursor was spin-coated onto a glass substrate. The desired asterisk shape was polymerized into the precursor by exposing the substrate with UV light through a printed transparency mask. After development, the SU-8 master was used to mold PDMS flow channels.

Two strategies were employed to change the direction of buffer flow through the separate channels and thus change the orientation of the plume. In the simpler design, five ports (in radial sequence) of the eight-port asterisk flow cell were connected to a Western Analytical port selection valve; the two ports on either side of the five were plugged and the final valve was coupled to a syringe pump. In the assembled flow cell the asterisk-shaped top flow cell and a single channel reagent flow cell (identical to that used in initial flow chambers) sandwiched the membrane. The multi-port flow cell was pumped from the port connected to the syringe pump while any of the five ports connected to the selection valve could be chosen to act as drain. By changing which port acted as drain, it was possible to change the directionality of flow in the culture chamber over ˜90°.

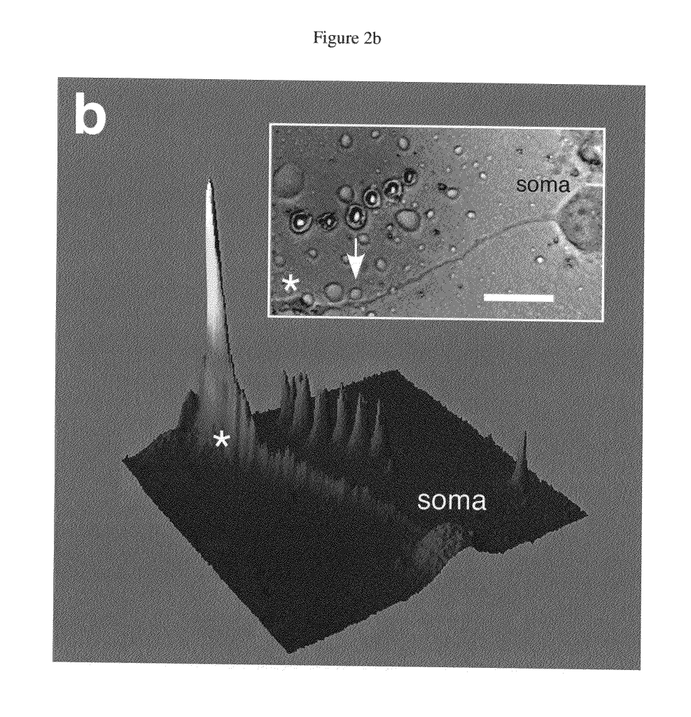

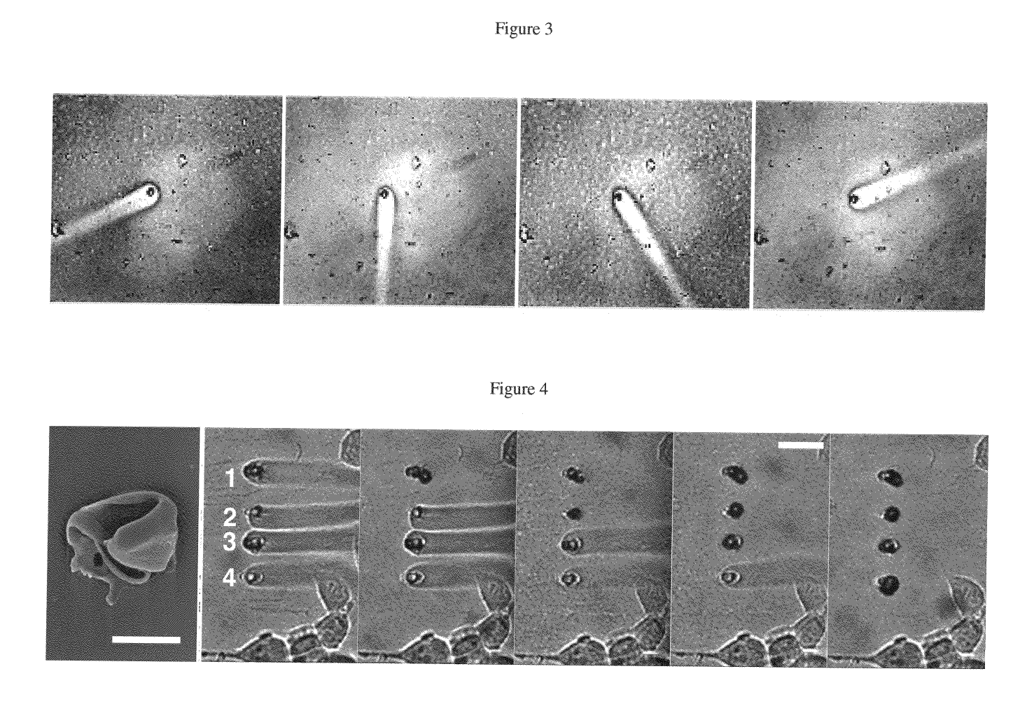

There were several disadvantageous to controlling the plume direction with a multi-port valve. One was that the plume could only be changed over a limited number of directions, and the other is that while the drain outlets changed the pumping inlet did not. This meant that the flow would have to change directions in the center of the channel, resulting in heterogenous linear flow rates at different locations in the central region of the flow cell, which would add complexity to predicting the orientation of a given plume. Another valving strategy sought to get around this issue. With this scheme the fluid connection at the ends of each of the four channels of the multi-port chamber was split into two ports so that the multi-port channel had 16 ports total. The one of the tubes leading from a port was paired with a tube from the other side of the channel and directed into pinch valve. After the pinch valve, one tube went to the syringe pump and the other went to the drain. The other pair of tubing had a similar arrangement, going through a pinch valve and connecting to the drain and to the syringe pump by means of a splitter. In terms of a single channel, this allowed the flow direction to be rapidly switched by simultaneously turning one pinch valve on and the other off. When all 16 ports were connected in such a manner it allowed flow in either direction in all four channels. With this set-up the flow did not change direction in the center of the channel, in principle making the plume orientation easier to predict. There was also the possibility that additional flow orientations could be achieved with this set-up by activating multiple ports at once. FIG. 3 demonstrates this valving scheme used to rapidly change plume orientation. The main problem during the trial of this system involved air in the tubing. Because portions of the tubing served to both feed and drain the channels, removing all the air out the system was critical. Otherwise air bubbles would move over the sample area or block the flow in channels. This however, proved challenging because of the extensive tubing required to drain and feed multiple the 16 ports of the multi-port channel.

Changing flow direction in this way also provides an effective way to rapidly turn on or off the chemical dosing of a desired cell or set of cells. For example, a reagent stream can be established in a manner that directs it away from cultured cells of interest. At a time of desired dosing, the directionality of flow will be rapidly altered such that the stream is directed at its intended target. Direction switching times will likely be on the millisecond time regime, though conceivably could be faster.

Open Top-Chamber Architecture for Mechanical Access

A major user of this invention will be of cell biologists (e.g., electrophysiologists), a group of scientists that generally demands the flexibility to interface with specimens both optically and mechanically. Consequently, it is useful to modify the chemical dosing system that was based on two fully enclosed flow chambers. By removing the top surface of the upper (cell-culture) flow cell, direct access to cultured cells is obtained, allowing experimenters to manipulate and analyze cells via mechanical means (e.g., using patch electrodes). Although it is difficult to model laminar-flow conditions under such environments, electrophysiologists as routine course employ open architecture flow chambers to rapidly exchange the solutions that expose cell cultures in low-Reynolds number regimes. Here, a variety of pump sources (e.g., multi-stage peristaltic pumps, syringes, syringe pumps) have been used to deliver solution to the flow inlet. In addition, because a truly enclosed flow cell geometry does not exist, fluid must be actively eliminated from the exit port using suction. In many instances, simple “house vacuum” systems are used for such purposes. We have demonstrated in proof-of-concept form that the open top-chamber geometry can be used with this chemical dosing procedure.

Termination of Flow Through Selected Pores

An ability to “valve-off” selected laminar streams while continuing to flow reagents through other pores would provide valuable on-the-fly control over the chemical landscapes that can be created with this approach. To eliminate flow through selected pores in cellular environments without use of complex, pre-fabricated microfluidics, we have adapted a biocompatible direct-write microfabrication procedure (19-21). In this approach, focused light from a pulsed laser (commonly a Ti:S oscillator) is used to promote intermolecular crosslinking of protein residue side-chains (e.g., tyrosine, cysteine, lysine) via three-dimensionally resolved multiphoton excitation of a photosensitizer (e.g., flavin adenine dinucleotide, FAD). Extended protein matrices with well defined three-dimensional topographies can be rapidly fabricated with feature sizes as small as ˜0.25 μm by scanning the laser focal point through desired solution coordinates. Unlike current direct-write procedures based on radical-initiated polymerization of synthetic organic monomers (22, 23), the aqueous, non-toxic reagents required for protein photocrosslinking allow protein microfabrication to be performed in biological microenvironments without disrupting cell viability (21).

In studies that evaluated protein photocrosslinking as a means to block membrane pores, we introduced a solution of 100 mg mL⁻¹ BSA and 5 mM FAD into the reagent chamber. By focusing ˜50 mW from the Ti:S laser (measured before the objective) onto the aperture entrance, BSA plugs (FIG. 4a) could be fabricated within less than 1 s that eliminated all apparent flow through a desired pore without visibly damaging the polymer membrane. FIG. 4b demonstrates a series in which plumes of BSA are sequentially eliminated in this manner. In addition, we have attempted to re-open pores by removing BSA plugs through a subsequent ablation event. Although pore re-opening is readily feasible, our preliminary studies have produced somewhat larger pores than were originally created in the membrane.

Cell Dosing Studies

For moderate solution flow rates, we found that flow had little or no effect on cells as assessed by adherence, morphology, and metabolic viability (see carboxyfluorescein diacetate studies, below). At flow rates above ˜0.5 mL min⁻¹ (˜0.8 cm s⁻¹ at a position 4 μm from the membrane) visible effects of shear stress could be observed on some NG108-15 cells plated on Mylar (e.g., minor deformation, partial lifting of neuritic structures) and at flow rates greater than 1.0 mL min⁻¹ a significant fraction of cells lost adherence within 10 min.

An initial assessment of the biocompatibility of the membrane ablation process was made by examining morphological properties of NG108-15 cells plated on Mylar during and after pore formation. Retraction of neurites and other signs of physical distress often are apparent when pores are made in direct proximity to cells (i.e., at spacings of less than 5 μm), but generally are not evident at somewhat greater distances (>10 μm). Provided that cells appear healthy immediately after ablation, they typically display no morphological degradation for experiments that last more than an hour. Were it advantageous to create a pore within 5 μm or less of a cell, the pressure differential between flow chambers could be reversed to minimize exposure of cells to membrane debris during pore formation. It also would be of value in future studies to evaluate transient temperature increases in the vicinity of a pore that accompany the ablation process.

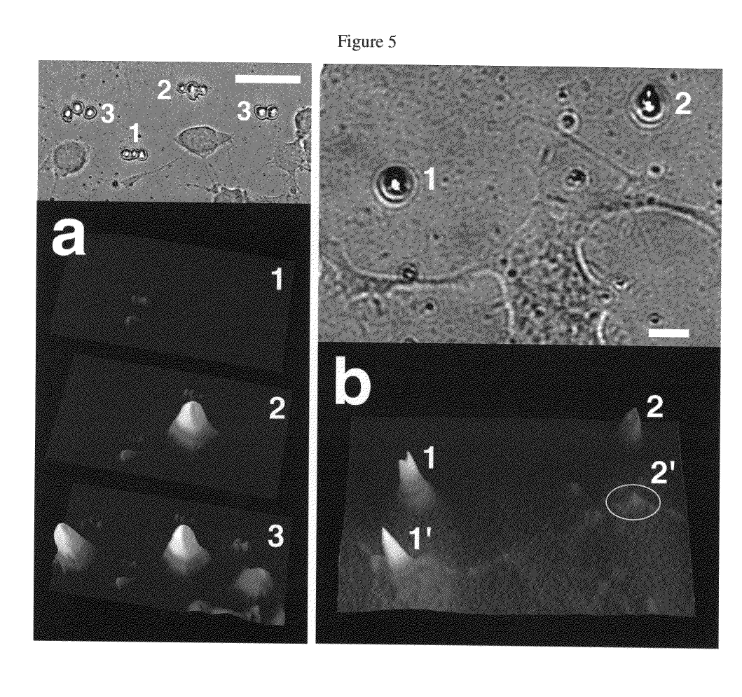

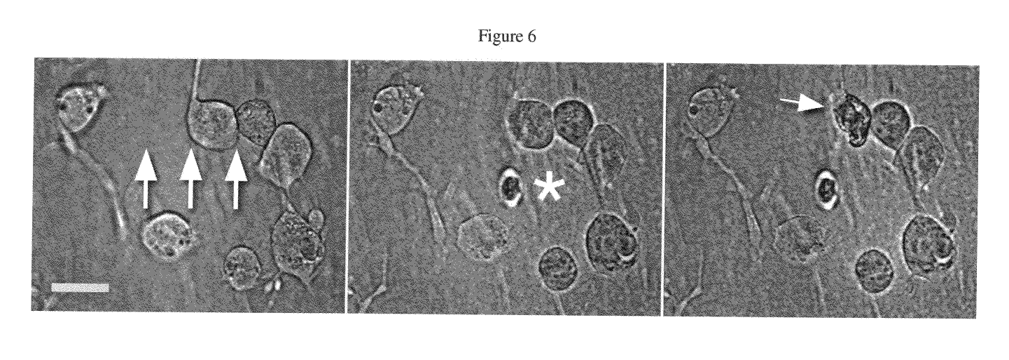

The ability to dose specific cells and subcellular regions with a labeling reagent was evaluated using 5-carboxyfluorescein diacetate (CFDA) (Molecular Probes, C1361), a membrane-permeable dye that is converted to its fluorescent form after hydrolysis by cytosolic esterases. Because visualization of this dye relies both on the continued functioning of metabolic components and a reasonably leak-free plasma membrane (24, 25), use of this dye served the additional role of further probing the viability of cells downstream of newly created pores. In these studies, 2.5-μm thick Mylar served as the barrier between flow chambers. To avoid occlusion of the pore by adherent bubbles, ablation was performed with the reagent chamber filled with air. Within several seconds of pore formation, CFDA was pumped into the upper flow cell, resulting in formation of a laminar-flow stream in the lower chamber directed at a selected target. CFDA was replaced with plain HBS buffer in the upper chamber after a dosing period of ˜1 min., and the procedure was repeated several times to sequentially dose targets at desired spatio-temporal coordinates. As shown in FIG. 5a, cellular features of specific interest—a neurite terminal and three cell bodies—could be directly targeted, as evidenced by an increase in fluorescein emission.

In general, we found that CFDA delocalized throughout the cytosol within minutes, a finding consistent with expected diffusion rates for this compound (26). For example, the neuritic fluorescence created by pore cluster 1 dissipated almost completely in the period between the initial CFDA exposure and dosing after creation of pore cluster 2 (data not shown). Only after CFDA flow was re-initiated did the neuritic structure once again become fluorescent.

We also found it feasible to selectively dose precise regions within cultured cells using Mitotracker Green FM (Molecular Probes, M7415), a dye that, at the concentrations used here, has been reported to localize to a variety of cellular structures. Because this dye is fluorescent principally when incorporated into lipid environments (e.g., organelles), cellular staining from this species delocalizes less rapidly than for cytosolic fluorophores with similar molecular masses, such as CFDA. To evaluate Mitotracker labeling, NG108-15 cells cultured on Mylar were dosed in a procedure similar to that used for CFDA except that the volumetric flow rate was increased to 0.45 mL min⁻¹. As shown in FIG. 5b, it is possible to target narrow regions (extending ˜10 μm) on two neurites on opposing sides of a cell body without appreciable staining of the soma itself

To test the ability of this system to selectively target cells for disruption, ethanol was flowed through the reagent chamber and a single pore was introduced near a cluster of cells plated on polycarbonate (FIG. 6). Within several seconds of pore formation, a cell positioned directly downstream of the pore undergoes extensive dehydration. The last (right-most) image in this sequence, taken ˜25 s after creation of the pore, shows that even after this relatively long period no additional cells within the field display signs of morphological damage.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

(1) Ito, H.; Nomoto, H.; Furukawa, Y.; Furukawa, S. Neuroscience Letters 2003, 339, 231-234.
(2) Contestabile, A.; Ciani, E. Neurochemistry International 2004, 45, 903-914.
(3) Karten, B.; Hayashi, H.; Campenot, R. B.; Vance, D. E.; Vance, J. E. Methods 2005, 36, 117-128.
(4) Brown, A. Nature Reviews Molecular Cell Biology 2000 1, 153-156.
(5) Zheng, J. Q.; Wan, J.; Poo, M. M. Journal of Neuroscience 1996, 16, 1140-1149.
(6) Wang, G. X.; Poo, M. M. Nature 2005, 434, 898-903.
(7) Matsuzaki, M.; Ellis-Davis, G.; Nemoto, T.; Miyashita, Y.; Kasai, H. nature neuroscience 2001, 4, 1086-1092.
(8) Callaway, E.; Yuste, R. Current Opinion in Neurobiology 2002, 12, 587-592.
(9) Peterman, M. C.; Noolandi, J.; Blumenkranz, M. S.; Fishman, H. A. Proceedings of the National Academy of Sciences of the United States of America 2004, 101, 9951-9954.
(10) Wu, H.; Huang, B.; Zare, R. N. Journal of the American Chemical Society 2006, 128, 4194-4195.
(11) Jeon, N. L.; Baskaran, H.; Dertinger, S. K. W.; Whitesides, G. M.; Van De Water, L.; Toner, M. Nature Biotechnology 2002, 20, 826-830.
(12) Taylor, A. M.; Rhee, S. W.; Tu, C. H.; Cribbs, D. H.; Cotman, C. W.; Jeon, N. L. Langmuir 2003, 19, 1551-1556.
(13) Takayama, S.; Ostuni, E.; LeDuc, P.; Naruse, K.; Ingber, D. E.; Whitesides, G. M. Nature 2001, 411, 1016.
(14) Takayama, S.; Ostuni, E.; LeDuc, P.; Naruse, K.; Ingber, D. E.; Whitesides, G. M. Chemistry & Biology 2003, 10, 123 -130.
(15) Duffy, D. C.; McDonald, J. C.; Schueller, O. J. A.; Whitesides, G. M. Anal. Chem. 1998, 70, 4974-4984.
(16) Abramoff, M. D.; Magelhaes, P. J.; Ram, S. J. Biophotonics Int. 2004, 11, 36-42.
(17) Joglekar, A. P.; Liu, H.; Spooner, G. J.; Mourour, G.; Hunt, A. J. Applied Physics B 2003, 77, 25-30.
(18) Schaffer, C. B.; Jamison, A. O.; Garcia, J. F.; Mazur, E. In Ultrafast Lasers; Sucha, G., Ed.; Marcel Dekker Inc.: New York, 2003, pp 395-418.
(19) Allen, R.; Nielson, R.; Wise, D.; Shear, J. Analytical Chemistry 2005, 77, 5089-5095.
(20) Hill, R.; Lyon, J.; Allen, R.; Stevenson, K.; Shear, J. Journal of the American Chemical Society 2005, 127, 10707-10711.
(21) Khaer, B.; Allen, R.; Javier, D.; Currie, J.; Shear, J. PNAS 2004, 101, 16104-16108.
(22) Cumpston, G. H.; Ananthavel, S. P.; Barlow, S.; Dyer, D. L.; Ehrlich, J. E.; Erskine, L. L.; Heikal, A. A.; Kuebler, S. M.; Lee, I.; McCord-Maughon, S. Nature 1999, 398, 51-54.
(23) Kawata, S.; Sun, H.; Tanaka, T.; Takada, K. Nature 2001, 412, 697-698.
(24) Satoh, T.; Sakai, S.; Kubo, T.; Enokido, Y.; Uchiyama, Y.; Hatanaka, H. Neuroscience Letters 1995, 201, 119-122.
(25) Provinciali, M.; Stefano, G. D.; Fabris, N. Journal of Immunological Methods 1992, 155, 19-24.
(26) Verrechia, F.; Duthe, F.; Duval, S.; Duchatelle, I.; Sarrouilhe, D.; Herve, J. C. Journal of Physiology 1999, 516, 447-459.

What is claimed is:

1. A system for site-specific dosing of cellular cultures, the system comprising:
at least two laminar flow channels; and
an ablatable membrane that is disposed between the at least two laminar flow channels.

2. The system of claim 1 wherein the ablatable membrane has been ablated by a directed energy source.

3. The system of claim 2 wherein the directed energy source is a femtosecond titanium/sapphire laser or a frequency-doubled Q-switched Nd:YAG laser.

4. The system of claim 1 wherein the ablatable membrane comprises polyethylene terephthalate, polycarbonate, or any derivative thereof.

5. The system of claim 1 wherein cells are cultured within at least one of the at least two laminar flow channels.

6. The system of claim 1 wherein at least one of the at least two flow channels is not fully enclosed.

7. The system of claim 1 further comprising a port selection valve connected to at least one of the at least two laminar flow channels.

8. A system for site-specific dosing of cellular cultures, the system comprising:
at least two laminar flow channels; and
an ablated membrane that is disposed between the at least two laminar flow channels.

9. The system of claim 8 wherein the ablated membrane has been ablated by a directed energy source.

10. The system of claim 8 wherein cells are cultured within at least one of the at least two laminar flow channels.

11. The system of claim 8 wherein at least one of the at least two flow channels is not fully enclosed.

12. The system of claim 8 further comprising a port selection valve connected to at least one of the at least two laminar flow channels.

13. A method for site-specific dosing of cellular cultures comprising the steps of:
providing at least two laminar flow channels;
providing a membrane disposed between the at least two laminar flow channels, wherein the membrane is selected from the group consisting of an ablatable membrane and an ablated membrane;
placing cells into at least one of the at least two laminar flow channels;
providing a reagent medium; and
flowing the reagent medium through at least one other of the at least two laminar flow channels.

14. The method of claim 13 wherein the membrane is an ablated polymer, wherein the ablated membrane has been ablated by a directed energy source.

15. The method of claim 13 further comprising the step of at least substantially reducing the transport of material across the ablatable or ablated membrane.

16. The method of claim 15 wherein the step of at least substantially reducing the transport of material across the ablatable or ablated membrane is performed with a directed energy source.

17. The method of claim 16 wherein the step of at least substantially reducing the transport of material across the ablatable or ablated membrane with a directed energy source comprises the crosslinking of proteins by the directed energy source.

18. The method of claim 14 wherein the directed energy source is a femtosecond titanium/sapphire laser or a frequency-doubled Q-switched Nd:YAG laser.

19. The method of claim 13 wherein at least one of the at least two flow channels is not fully enclosed.

20. The method of claim 13 further comprising the steps of:
providing a port selection valve;
connecting the port selection valve to at least one of the at least two laminar flow channels; and
allowing the port selection valve to control the flow of fluid through at least one of the at least two laminar flow channels.
